(19) United States
(12) Patent Application Publication
(10) Pub. No.: US 2018/0049958 A1
(43) Pub. Date: Feb. 22, 2018

(54) AGENT FOR IMPROVED OXIDATIVE LIGHTENING OF KERATINIC FIBRES

(71) Applicant: Henkel AG & Co. KGaA, Duesseldorf (DE)

(72) Inventors: Udo Erkens, Willich (DE); Burkhard Mueller, Duesseldorf (DE)

(73) Assignee: Henkel AG & Co. KGaA, Duesseldorf (DE)

(21) Appl. No.: 15/664,117

(22) Filed: Jul. 31, 2017

(30) Foreign Application Priority Data
Aug. 16, 2016 (DE) 10 2016 215 198.3

(51) Int. Cl. A61K 8/22; A61K 8/23; A61K 8/55; A61K 8/41; A61Q 5/08

(52) U.S. Cl. CPC A61K 8/22; A61K 8/23; A61K 2800/882; A61K 8/41; A61Q 5/08; A61K 8/55

(57) ABSTRACT

Agents and methods for lightening keratinic fibres are provided herein. In an embodiment, agents for lightening keratinic fibres include at least two separately packaged preparations (A) and (B) as well as an optional further preparation (C) which is packaged separately from preparations (A) and (B). Preparations (A), (B), and, when present, preparation (C) are mixed immediately prior to use in order to form a mixture for application. Preparation (A) includes at least one persulphate and at least two chelating agents. Preparation (B) is free-flowing and includes at least one oxidizing agent.

### CROSS-REFERENCE TO RELATED APPLICATION

This application claims priority to German Patent Application No. DEI02016215198.3, filed Aug. 16, 2016 which is incorporated herein by reference in its entirety.

### TECHNICAL FIELD

The present disclosure pertains to agents for the oxidative modification of color in the field of cosmetics, which are particularly suitable for lightening keratinic fibres, in particular human hair.

### BACKGROUND

Modifying the shape and color of the hair constitutes an important field of modern cosmetics. In addition to dyeing, lightening of the actual hair fibres or bleaching them blond is a particular wish for many consumers, because a blond hair color is considered to be attractive and desirable from a fashion viewpoint. Various bleaching agents with a variety of bleaching powers are available on the market.

The oxidizing agents contained in bleaching agents are capable of lightening hair fibres by oxidatively destroying the hair's own pigment, namely melanin. Using hydrogen peroxide as the only oxidizing agent is appropriate for a moderate bleaching effect—possibly with the addition of ammonia or other alkalizing agents—but in order to obtain a stronger bleaching effect, a mixture of hydrogen peroxide and peroxodisulphate salts and/or peroxomonosulphate salts is used.

For reasons of stability, commercial bleaching agents are usually offered for sale in two separately packaged preparations which are mixed together immediately prior to use to form a ready-to-use preparation. Usually, commercial bleaching agents includes a liquid oxidizing agent preparation and a powder which contains solid oxidizing agents.

For bleaching, the powder with the solid oxidizing agents is mixed with the hydrogen peroxide solution immediately prior to use. This mixture is then applied to the hair and is then rinsed out after a specific treatment time. In order to obtain a sufficient bleaching effect, agents of this type are usually highly alkaline, with a pH of between about 9 and about 10.5. Values this high for the pH are necessary in order to ensure that the outer cuticle layer opens up, thus allowing the active species (hydrogen peroxide and persulphate) to penetrate into the hair.

When the hair starts off being dark, longer application times and/or repeated bleaching procedures are usually required in order to lighten the hair. However, this is associated with more severe damage to the hair, because not only the pigments of the hair, but also the other structural components of the hair are oxidatively damaged. The severity of the damage can range from rough, brittle and hard-to-comb hair to compromised resistance and tensile strength of the hair, and even to breakage of the hair.

The use of chelating agents during the oxidationive color modification of keratinic fibres is known. The chelating agents are aimed, inter alia, at preventing the decomposition of the hydrogen by metal ions concentrated in the hair fibres.

Thus, EP 1 714 634 A1 describes a hair treatment kit for coloring human hair, comprising a first compartment which contains a chelating agent, and a second component which contains coloring agents. By using a chelating agent, unwanted reactions on and with hair, leading to unwanted heating, can be avoided.

Thus, the aim of the present disclosure is to mitigate the disadvantages which have been mentioned of known oxidative bleaching agents. In particular, there is a need to provide elements containing oxidizing agents for lightening keratinic fibres with an enhanced lightening power.

### BRIEF SUMMARY

Agents and methods for lightening keratinic fibres are provided herein. In an embodiment, agents for lightening keratinic fibres include at least two separately packaged preparations (A) and (B) as well as an optional further preparation (C) which is packaged separately from preparations (A) and (B). Preparations (A), (B), and, when present, preparation (C) are mixed immediately prior to use in order to form a mixture for application. Preparation (A) includes at least one persulphate and at least two chelating agents. Preparation (B) is free-flowing and includes at least one oxidizing agent.

In another embodiment, a method for lightening keratinic fibres is provided. In accordance with the method, at least two separately packaged preparations (A) and (B) are mixed together to form a mixture for application. The mixture is applied to the fibres. After a treatment time the mixture is rinsed out again. Preparation (A) includes at least one persulphate and at least two chelating agents, and preparation (B) comprises at least one oxidizing agent.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

This aim is achieved by employing agents for lightening keratinic fibres, containing at least two separately packaged preparations (A) and (B) as well as an optional further preparation (C) which is packaged separately from (A) and (B), which are mixed immediately prior to use in order to form a mixture for application, wherein
i. preparation (A) contains at least one persulphate, and
ii. preparation (B) is free-flowing and contains at least one oxidizing agent, and the preparation (A) furthermore contains at least two chelating agents.

The term “keratinic fibres” or even “keratin fibres” should be understood to mean fur, wool, feathers and in particular human hair. Although the agents are primarily suitable for lightening keratinic fibres, in principle, usage in other fields as well is not excluded.

The preparations (A) are preferably in powder form. In this regard, powders formed from solid components with different grain sizes may be employed. Normally, it is preferable for the powder, however, to have as homogeneous a grain size as possible, in particular in order to make it easier to obtain a uniform dispersion or solution of the powder in the preparations (B).

The preparations (A) may contain the substances in a solid cosmetic support. A solid cosmetic support may contain silica salts, in particular silicate and metasilicate salts with ammonium, alkali metals or alkaline-earth metals. In particular, metasilicates, with formula (SiO₂)n(M₂O)ₘ, wherein M represents an ammonium ion, an alkali metal or a half-stoichiometric equivalent of an alkaline-earth metal, are exemplified by the ratio between n and m of ≦ about 1 and can be construed as being chain-like polymeric structures of the anion [SiO₃]²⁻, may preferably be employed. Sodium metasilicate with formula [Na₂SiO₃]_{∞} is particularly preferred in this regard. Equally preferred as those silicates which are formed by a silicate with formula (SiO₂)ₙ(Na₂O)ₘ(K₂O)ₚ, wherein n represents a positive rational number and m and p, independently of each other, represent a positive rational number or 0, with the proviso that at least one of the parameters m or p is other than 0 and that the ratio between n and the sum of m and p is between about 2:1 and about 4:1.

Furthermore, the solid cosmetic supports may contain what are known as anticaking agents, which are intended to prevent clumping or caking of the powdered components. Preferred anticaking agents of this type are water-insoluble, hydrophobic or moisture-adsorbing powders of diatomaceous earth, pyrogenic silicas, calcium phosphate, calcium silicates, aluminium oxide, magnesium oxide, magnesium carbonate, zinc oxide, stearates, fatty amines and the like.

Finally, the solid cosmetic supports may additionally contain a dust control agent which prevents the powdered components from forming dust. In this regard, in particular, inert oils may be used. Preferably, the solid cosmetic supports contain ester oils or mineral oils, preferably hydrocarbon oils such as liquid paraffin oil, as the dust control agent.

The preparation (A) contains at least one persulphate salt as the first essential ingredient. Suitable persulphate salts are inorganic peroxy compounds. These are preferably selected from ammonium peroxodisulphate, alkali metal peroxodisulphates, ammonium peroxomonosulphate, alkali metal peroxomonosulphates, alkali metal peroxodiphosphates and/or alkaline-earth metal peroxides. Ammonium peroxodisulphate and/or alkali metal peroxodisulphates are particularly preferred.

In a preferred embodiment of the present disclosure, preparation (A) contains at least one peroxy disulphate salt as the persulphate salt, in particular selected from ammonium peroxodisulphate and/or potassium peroxodisulphate and/or sodium peroxodisulphate.

Furthermore, it has been shown to be particularly preferable for the preparations (A) to contain at least two different peroxodisulphates. Preferred peroxodisulphate salts in this regard are combinations of ammonium peroxodisulphate with potassium peroxodisulphate and/or sodium peroxodisulphate.

The preparations (A) contain persulphate salts, preferably in a quantity of from about 0.1 % to about 80 % by weight, preferably from about 2 % to about 60 % by weight, particularly preferably from about 3 % to about 50 % by weight and more particularly preferably from about 5 % to about 45 % by weight, respectively with respect to the total weight of preparation (A).

Preparation (A) contains at least two chelating agents as the further imperative ingredient. The at least two chelating agents are preferably selected from the group of
(a) nitrilotriacetic acid (NTA),
(b) diethylenetriamine pentaacetic acid (DTPA),
(c) ethylenediamine disuccinic acid (EDDS),
(d) ethylenediamine diglutaric acid (EDGA),
(e) 2-hydroxypropylenediamine disuccinic acid (HPDS),
(f) glycinamide-N,N′-disuccinic acid (GADS),
(g) ethylenediamine-N-N′-diglutaric acid (EDDG),
(h) 2-hydroxypropylenediamine-N-N′-disuccinic acid (HPDDS),
(i) ethylenediamine tetraacetic acid (EDTA),
(j) ethylenedicysteic acid (EDC),
(k) diaminoalkyl di(sulphosuccinic acid) (DDS),
(l) ethylenediamine-N-N′-bis(ortho-hydroxyphenyl acetic acid (EDDHA),
(m) N-2-hydroxyethyl-N,N-diacetic acid,
(n) glycerylimino diacetic acid,
(o) iminodiacetic acid-N-2-hydroxypropylsulphonic acid,
(p) aspartic acid-N-carboxymethyl-N-2,5-hydroxypropyl-3-sulphonic acid,
(q) α-alanine-N,N′-diacetic acid,
(r) aspartic acid-N,N′-diacetic acid,
(s) aspartic acid-N-monoacetic acid,
(t) dipicolinic acid,
(u) 1-hydroxyethane-(1,1-diphosphonic acid) (HEDP) as well as
(v) salts and derivatives thereof.

Regarding the lightening power, the use of a salt, preferably a sodium salt, ethylenediamine tetraacetic acid (EDTA), has proved to be particularly advantageous having regard to preparation (A). Examples of suitable EDTA salts include disodium ethylenediamine tetraacetate (Na₂H₂EDTA), tetrasodium ethylenediamine tetraacetate (Na₄EDTA) and calcium disodium ethylenediamine tetraacetate (CaNa₂EDTA), wherein disodium ethylenediamine tetraacetate is preferred. Preferably again, preparation (A) contains the salt, preferably a sodium salt, of ethylenediamine tetraacetic acid (EDTA) and at least one further chelating agent.

1-hydroxyethane-(1,1-diphosphonic acid) (HEDP) is also a preferred chelating agent. Correspondingly, preparation (A) preferably contains 1-hydroxyethane-(1,1-diphosphonic acid) and at least one other chelating agent.

In a particularly preferred embodiment, preparation (A) contains a salt, preferably a sodium salt, of ethylene diamine tetraacetic acid (EDTA), and 1-hydroxyethane-(1,1-diphosphonic acid) as chelating agents.

More preferably, the weight ratio of the salt, preferably the sodium salt, of ethylene diamine tetraacetic acid (EDTA) to the 1-hydroxyethane-(1,1-diphosphonic acid) is from about 5:1 to about 1:5, more preferably from about 2:1 to about 1:2, and particularly preferably about 1:1.

In a most particularly preferred embodiment, preparation (A) contains, as the chelating agent, disodium ethylenediamine tetraacetate and 1-hydroxyethane-(1,1-diphosphonic acid) in a weight ratio of 1:1.

In a more particularly preferred embodiment, preparation (A)—with respect to its weight—contains the at least two chelating agents in a total quantity of from about 0.02 % to about 12 % by weight, more preferably from about 0.1 % to about 6 % by weight, and more particularly preferably from about 0.2 % to about 3 % by weight.

It has surprisingly been shown that when using at least two chelating agents, the lightening power is greater than when using only one chelating agent. In addition, an improved stability on storage of preparation (A), in particular by preventing or slowing down the decomposition of the persulphate salt, has been observed.

The preparations (B) and optionally (C) contain the substances in a free-flowing cosmetic support. The base for the free-flowing cosmetic support is thus preferably aqueous or hydro-alcoholic. For the purposes of bleaching the hair, supports of this type, for example, gels or, in addition, surfactant-containing foaming solutions such as, for example, shampoos, foaming aerosols or other preparations which are suitable for application to the hair, are suitable. A preferred free-flowing support as contemplated herein contains at least about 40 % by weight, in particular at least about 50 % by weight of water. The term “hydro alcoholic supports” as used in the context of the present disclosure should be understood to mean water-containing compositions containing from about 3 % to about 70 % by weight of a C₁-C4 alcohol, in particular ethanol or isopropanol. The preparations (B) and optionally (C) may respectively additionally contain other organic solvents such as, for example, methoxybutanol, ethyldiglycol, 1,2-propylene glycol, n-propanol, n-butanol, n-butyleneglycol, glycerine, diethyleneglycol monoethylether, and diethyleneglycol mono-n-butylether. In this regard, any water-soluble organic solvent is preferred. Preferably, preparation (B) is a liquid.

Preparations (B) contain hydrogen peroxide as the oxidizing agent.

The concentration of a hydrogen peroxide solution in preparation (B) is determined on the one hand by legal requirements and on the other hand by the desired effect.

Preferably, the preparations (B), with respect to their total weight, contain hydrogen peroxide in quantities of from about 0.5 % to about 30 % by weight, preferably from about 1 % to about 20 % by weight, particularly preferably from about 5 % to about 15 % by weight and more particularly preferably from about 6 % to about 12 % by weight, or explicitly about 6 %, about 7 %, about 8 %, about 9 %, about 10 %, about 11 % or about 12 % by weight.

Preferred ready-to-use agents contain, with respect to the total weight of the ready-to-use agent, from about 0.01 % to about 12 % by weight, preferably from about 0.1 % to about 10 % by weight, particularly preferably from about 3 % to about 9 % by weight of hydrogen peroxide.

The ready-to-use agents for lightening keratinic fibres are produced immediately prior to application to the hair by mixing the two preparations (A) and (B) and optionally a third preparation (C) and/or further preparations. The consistency of the ready-to-use agents spans from free-flowing to spreadable.

The viscous properties of preparation (B) are of importance as regards good miscibility and high stability. The preparations (B) thus preferably have a viscosity of about 1000 mPa·s to about 50000 mPa·s, preferably from about 5000 mPa·s to about 45000 mPa·s and particularly preferably from about 7000 mPa·s to about 40000 mPa·s, the measurements being made with a rotational viscosimeter from Brookfield, spindle size 4, at 25° C. and 4 rpm. The ready-mixed and ready-to-use agents preferably have a viscosity of from about 10000mPa·s to about 100000 mPa·s and particularly preferably from about 18000 mPa·s to about 80000 mPa·s, the measurements being made with a rotational viscosimeter from Brookfield, spindle size 5, at 25° C. and 4 rpm.

The agents may contain further substances and auxiliary substances. These shall be described below.

It may be advantageous for preparation (B) to contain at least one non-ionic surfactant, preferably at least one ethoxylated fatty alcohol containing from about 40 to about 60ethylene oxide units. This should be understood to include an addition product of ethylene oxide and a fatty alcohol. Fatty alcohols in this regard are saturated and unsaturated alcohols containing 12 to 24 C atoms, which may be linear or branched. The molar quantity of ethylene oxide used per mole of fatty alcohol describes the degree of ethoxylation. Particularly suitable non-ionic surfactants in this regard are addition products of ethylene oxide with octyl alcohol (capryl alcohol), nonyl alcohol (pelargonyl alcohol), undecyl alcohol, undec-10-en-1-ol, dodecyl alcohol (lauryl alcohol), 2,6,8-trimethyl-4-nonanol (isolauryl alcohol), tridecyl alcohol, tetradecyl alcohol (myristyl alcohol), pentadecyl alcohol, hexadecyl alcohol (cetyl-/palmityl alcohol), heptadecyl alcohol, octadecyl alcohol (stearyl alcohol), isostearyl alcohol, (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenyl alcohol), nonadecan-1-ol (nonadecyl alcohol), eicosan-1-ol (eicosyl alcohol/arachyl alcohol), (9Z)-eicos-9-en-1-ol (gadoleyl alcohol), (5Z,8Z,11Z,14Z)-eicosa-5,8,11,14-tetraen-1-ol (arachidonic alcohol), heneicosyl alcohol, docosyl alcohol (behenyl alcohol), (13Z)-docos-13-en-1-ol (erucyl alcohol) or (13E)-docosen-1-ol (brassidyl alcohol). It is also possible to use mixtures of fatty alcohols which are obtained by specific mixing or in fact by extraction processes per se. Examples are cocosalcohol (mixture of C₈-C₁₈ fatty alcohols) or cetearyl alcohol (1:1 mixture of C₁₆ and C₁₈ fatty alcohols).

Degrees of ethoxylation of from about 20 to about 60 are preferred. Preferred non-ionic surfactants of the fatty alcohol type are ceteareth-20 and ceteareth-50.

Furthermore, setting the pH of the ready-mixed and ready-to-use agent is of importance for the lightening power. Values for the pH of between about 9 and about 12 are preferred for the ready mixed and ready-to-use agents.

Furthermore, in this regard, the agents may contain alkalizing agents. Examples of preferred alkalizing agents are ammonia, alkanolamines, basic amino acids, as well as inorganic alkalizing agents such as alkali or alkaline-earth metal hydroxides, alkali or alkaline-earth metal metasilicates, alkali or alkaline-earth metal silicates, alkali or alkaline-earth metal phosphates and alkali or alkaline-earth metal hydrogen phosphates. Preferred metal ions are lithium, sodium and/or potassium. Preferred alkalizing agents are alkali or alkaline-earth metal metasilicates and alkali or alkaline-earth metal silicates.

Suitable inorganic alkalizing agents are preferably selected from sodium hydroxide, potassium hydroxide, calcium hydroxide, barium hydroxide, sodium phosphate, potassium phosphate, sodium silicate, potassium silicate, magnesium silicate, sodium carbonate and potassium carbonate. Sodium hydroxide and/or potassium hydroxide are particularly preferred.

Alkanolamine alkalizing agents are preferably selected from primary, secondary or tertiary amines with a C₂-C₆ alkyl backbone which carries at least one hydroxyl group. Particularly preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol (monoisopropanolamine), 1-aminobutan-2-ol, 1-aminopentan-2-ol, 1-aminopentan-3-ol, 1-aminopentan-4-ol, 2-amino-2-methyl-propanol, 2-amino-2-methylbutanol, 3-amino-2-methylpropan-1-ol, 1-amino-2-methylpropan-2-ol, 3-aminopropan-1,2-diol, 2-amino-2-methylpropan-1,3-diol, 2-amino- 2-ethyl-1,3-propanediol, N,N-dimethylethanolamine, methylglucamine, triethanolamine, diethanolamine and triisopropanolamine. Particularly preferred alkanolamines are monoethanolamine, 2-amino-2-methyl-propanol and triethanolamine.

Preferred amino acids for use as the alkalizing agent are selected from the group formed by L-arginine, D-arginine, D/L-arginine, L-lysine, D-lysine, D/L-lysine, L-ornithine, D-ornithine, D/L-ornithine, L-histidine, D-histidine and/or D/L-histidine. L-arginine, D-arginine and/or D/L-arginine are particularly preferable for use as alkalizing agents.

Furthermore, it has been shown to be advantageous for the preparation (B) to contain at least one stabilizer or chelating agent.

Particularly preferred stabilizers are phenacetin, alkali benzoate (sodium benzoate) and salicylic acid.

Suitable chelating agents which may be contained in the preparation (B) comprise:
a) polycarboxylic acids, in which the sum of the carboxyl and optional hydroxyl groups is at least 5, such as gluconic acid;
b) nitrogen-containing mono- or polycarboxylic acids such as ethylenediamine tetraacetic acid (EDTA), N-hydroxyethylethylene diaminotriacetic acid, diethylene triaminopentaacetic acid (DTPA), ethylenediamine disuccinic acid (EDDS), hydroxyethylimino diacetic acid, nitridodiacetic acid-3-propionic acid, isoserine diacetic acid, N,N-di-(2-hydroxyethyl)glycine, N-(1,2-dicarboxy-2-hydroxyethyl)glycine, N-(1,2-dicarboxy-2-hydroxyethyl)aspartic acid or nitrilotriacetic acid (NTA), ethylenediamine diglutaric acid (EDGA), 2-hydroxypropylenediamine disuccinic acid (HPDS), glycinamide-N,N′-disuccinic acid (GADS), ethylenediamine-N-N′-diglutaric acid (EDDG), 2-hydroxypropylenediamine-N-N′-disuccinic acid (HPDDS), diaminoalkyldi-(sulphosuccinic acid) (DDS), ethylenedicysteic acid (EDC), ethylenediamine-NN′-bis(orthohydroxyphenyl)acetic acid (EDDHA), N-2-hydroxyethylamino-N,N-diacetic acid, glyceryliminodiacetic acid, iminodiacetic acid-N-2-hydroxypropylsulphonic acid, aspartic acid-N-carboxymethyl-N-2,5-hydroxypropyl-3-sulphonic acid, α-alanin-N,N′-diacetic acid, aspartic acid-N,N′-diacetic acid, aspartic acid-N-monoacetic acid, dipicolinic acid, as well as their salts and/or derivatives;
c) geminal diphosphonic acids such as 1-hydroxyethane-1,1-diphosphonic acid (HEDP), its higher homologues containing up to 8 carbon atoms, as well as derivatives containing hydroxyl or amino groups and 1-aminoethane-1,1 -diphosphonic acid, its higher homologues containing up to 8 carbon atoms as well as derivatives containing hydroxyl or amino group;
d) aminophosphonic acids such as ethylenediamine tetra(methylenephosphonic acid) (EDTMP), di-ethylene-triaminopenta(methylenephosphonic acid) (DTPMP) as well as its higher homologues, or nitrilo(methylenephosphonic acid);
e) phosphonopolycarboxylic acids such as 2-phosphonobutane-1,2,4-tricarbonic acid, and/or
f) cyclodextrin.

The preparations (A) and (B) may be mixed with other separately packaged preparations immediately before application, to form a mixture for application.

In a preferred embodiment, the agent additionally contains at least one further preparation (C) which is packaged separately from the preparations (A) and (B), wherein preparation (C) contains at least one alkalizing agent.

Irrespective of whether preparation (C) and/or preparation (B) and/or other preparations contain alkalizing agents, when alkalizing agents are employed, those agents are preferred which contain alkalizing agents in a quantity of from about 0.05 % to about 20 % by weight, in particular fromn about 0.5 % to about 10 % by weight, respectively with respect to the total weight of the entire ready-to-use agent.

To further increase the lightening power, to reinforce the bleaching, preparation (C) may additionally be supplemented with a silicon-containing compound. This is preferably selected from the group formed by silica, alkali metal silicates and alkaline-earth metal silicates.

Particularly preferably, the lightening or bleaching agent contains specific direct dyes in the complementary colors in order to tone down unwanted residual color nuances, in particular in the red or blue regions. These are dyes which act directly on the hair and do not require any oxidative processes in order to form the color. Direct dyes are usually nitrophenylenediamines, nitroaminophenols, azo dyes, anthraquinones or indophenols. Direct dyes are known as anionic, cationic and non-ionic direct dyes. The direct dyes are each preferably used in a quantity of from about 0.001 % to about 2 % by weight, with respect to the entire preparation for application.

Furthermore, the agents may contain other substances, auxiliary substances and additives such as, for example, non-ionic polymers, cationic polymers, anionic polymers, zwitterionic and amphoteric polymers, structuring agents, thickening agents, hair conditioning compounds, fiber structure-improving substances, other surfactants, colorants for coloring the agent, anti-dandruff substances, amino acids, oligopeptides, protein hydrolysates on an animal and/or plant bases, light stabilizers, UV blockers, vitamins, provitamins, vitamin precursors, plant extracts, fats, waxes, swelling and penetration substances, opacifiers, pearlescent agents, thickeners and pigments.

In a second aspect, the present disclosure provides a method for modifying the color of keratinic fibres, wherein at least two separately packaged preparations (A) and (B), of which preparation (A) contains at least one persulphate, and at least two chelating agents and preparation (B) contains at least one oxidizing agent, are mixed to form a mixture for application, which is applied to the fibres and is rinsed out again after a treatment time.

The ready-to-use agents are produced immediately prior to application to the hair by mixing the two preparations (A) and (B) and optionally a third preparation (C) and/or further preparations. In ready-to-use agents which are mixed from more than two preparations to form a prepared mixture for application, it may be immaterial whether initially two preparations are mixed together and then the third preparation is added and mixed in, or whether all of the preparations are placed together at the same time and then mixed. Mixing may be carried out by stirring in a bowl or dish or by shaking in a closable container.

The term “immediately” in this regard should be understood to mean a time period of a few seconds to about one hour, preferably to about 30 min, in particular to about 15 min.

The agents are applied in a method for lightening keratinic fibres, in particular human hair, in which the agent is applied to the keratinic fibres, left on the fibres for a treatment time of from about 10 to about 60 minutes and then rinsed out again with water or washed out with a shampoo.

Preferably, the treatment time for the ready-to-use lightening agent is from about 10to about 60 min, in particular from about 15 to about 50 min, particularly preferably from about 20 to about 45 min. During the treatment time for the agent on the fibres, it may be advantageous to assist the lightening process by adding a little heat. The heat may be added by employing an external heat source, such as with the aid of a flow of warm air, and also, in particular when lightening the hair of living subjects, by employing the body temperature of the subject. In the latter case, the part to be lightened is usually covered with a hood. A work phase at ambient temperature is preferred.

After the end of the treatment time, the remaining lightening preparation is rinsed out of the hair with water or a cleansing agent. In particular, the cleansing agent may be a commercial shampoo, but in particular, a cleansing agent can be dispensed with and the rinsing procedure can be carried out with mains water if the lightening agent contains a support with a high surfactant content.

The preferred embodiments of the agent described above apply to the method mutatis mutandis.

In a further aspect, the present disclosure provides the use of at least two chelating agents in a preparation (A), which is a component for an agent for lightening keratinic fibres, containing at least two separately packaged preparations (A) and (B) as well as, optionally, a preparation (C) which is packaged separately from (A) and (B) which are mixed immediately prior to application in order to form a mixture for application, wherein
i. preparation (A) contains at least one persulphate, and
ii. preparation (B) is free-flowing and contains at least one oxidizing agent, in order to increase the lightening power when lightening keratinic fibres.

The preferred embodiments of the agent described above also apply to the use mutatis mutandis.

In a further aspect, the present disclosure provides a method for increasing the lightening power when lightening keratinic fibres, in which at least two chelating agents of a preparation (A), which is a component of an agent for lightening keratinic fibres, containing at least two separately packaged preparations (A) and (B) as well as, optionally, a further preparation (C) which is packaged separately from (A) and (B), which are mixed immediately prior to use in order to form a mixture for application, wherein
i. preparation (A) contains at least one persulphate, and
ii. preparation (B) is free-flowing and contains at least one oxidizing agent.

The following examples provide more details of the present disclosure without in any way limiting its scope.

### EXAMPLES

TABLE 1
Composition of preparation (A) for bleaching agent
formed using two preparations (as a % by weight)

| Ingredient | A |
|---|---|
| Na₂H₂EDTA | 0-6 |
| HEDP | 0-3 |
| Sodium hexametaphosphate | 0.3 |
| Sodium metasilicate | 18 |
| Aerosil 200 | 2 |
| Rohagit S hv | 2 |
| Cekol 50000 | 2.5 |
| Ammonium persulphate + 0.5% silica | 10.0 |
| Potassium persulphate | 32 |
| Paraffinum Liquidum | 6 |
| Fragrance | 0.2 |
| Calcium stearate | 0.5 |
| Starch | 2 |
| Colorant | 0.5 |
| Magnesium carbonate and water | qs 100 |

*Starting materials used: Aerosil 200 (INCI name: Silica (Evonik Degussa)), Rohagit S hv (INCI name: Acrylates Copolymer (Evonik Röhm)), Cekol 50000 (INCI name: Cellulose Gum (CP Kelco))

A variety of preparations (A) were prepared using different quantities of chelating agents Na₂H₂EDTA and HEDP.

TABLE 2
Quantity of chelating agents in the respective preparations (A) for
bleaching agent formed using two preparations (as a % by weight)

| Preparation | Na₂H₂EDTA | HEDP |
|---|---|---|
| (A)-V0 | — | — |
| (A)-V1 | 1 | — |
| (A)-E1 | 0.5 | 0.5 |
| (A)-V2 | 2 | — |
| (A)-E2 | 1 | 1 |
| (A)-V3 | 3 | — |
| (A)-E3 | 1.5 | 1.5 |
| (A)-V4 | 6 | — |
| (A)-E4 | 3 | 3 |

TABLE 3
Composition of preparation (B) for bleaching agent
formed from two preparations (as a % by weight)

| Ingredient | B |
|---|---|
| Potassium hydroxide solution (50%) | 0.2 |
| Dipicolinic acid | 0.1 |
| Disodium pyrophosphate | 0.1 |
| HEDP 60% | 0.25 |
| Hydrogen peroxide (50%) | 12.2 |
| Paraffin | 2 |
| Cetearyl Alcohol | 4 |
| Water, demineralized | qs 100 |

To produce a bleaching agent, in each case one of the preparations (A)-VO to (A)-E4 (see Table 2) was mixed with the preparation (B) in a ratio of 1:2.

Different strands of hair (Kerling natural European hair 3-0, 10 cm in length) were treated for 45 minutes with a bleaching agent based on preparations (A)-V0 to (A)-E4 and dried. In each case, 3 strands of hair were produced and measured colorimetrically (Datacolor Spectraflash SF 450). The bleaching procedure was sometimes carried out up to three times.

TABLE 4
CIE Lab values:

| Degree of bleaching/Preparation (A) | CIE L | CIE a | CIE b |
|---|---|---|---|
| unbleached/— | 18.7 | 3.9 | 4.1 |
| 1 x bleached/(A)-V0 | 45.5 | 12.8 | 30.1 |
| 1 x bleached/(A)-V1 | 48.8 | 12.9 | 31.7 |
| 1 x bleached/(A)-E1 | 52.8 | 12.2 | 31.9 |
| 1 x bleached/(A)-V2 | 49.0 | 12.8 | 31.9 |
| 1 x bleached/(A)-E2 | 54.0 | 12.1 | 33.0 |
| 1 x bleached/(A)-V3 | 51.6 | 12.6 | 32.7 |
| 1 x bleached/(A)-E3 | 54.4 | 12.4 | 33.3 |
| 1 x bleached/(A)-V4 | 51.1 | 12.6 | 32.4 |
| 1 x bleached/(A)-E4 | 60.4 | 10.8 | 33.9 |
| 2 x bleached/(A)-V0 | 60.9 | 10.0 | 31.9 |
| 2 x bleached/(A)-V1 | 61.6 | 10.0 | 32.2 |
| 2 x bleached/(A)-E1 | 67.1 | 8.3 | 30.2 |
| 2 x bleached/(A)-V2 | 63.0 | 9.2 | 31.3 |
| 2 x bleached/(A)-E2 | 65.6 | 8.6 | 30.9 |
| 2 x bleached/(A)-V3 | 64.8 | 8.6 | 30.8 |
| 2 x bleached/(A)-E3 | 69.5 | 7.5 | 29.9 |
| 2 x bleached/(A)-V4 | 66.9 | 8.1 | 30.2 |
| 2 x bleached/(A)-E4 | 71.8 | 5.7 | 28.4 |
| 3 x bleached/(A)-V0 | 71.8 | 5.6 | 26.2 |
| 3 x bleached/(A)-V1 | 73.2 | 5.0 | 25.5 |
| 3 x bleached/(A)-E1 | 74.2 | 3.9 | 24.0 |
| 3 x bleached/(A)-V2 | 75.9 | 4.1 | 24.4 |
| 3 x bleached/(A)-E2 | 74.9 | 3.8 | 23.0 |
| 3 x bleached/(A)-V3 | 74.2 | 3.5 | 23.2 |
| 3 x bleached/(A)-E3 | 76.6 | 3.4 | 22.6 |
| 3 x bleached/(A)-V4 | 75.1 | 3.3 | 22.3 |
| 3 x bleached/(A)-E4 | 77.1 | 1.9 | 19.6 |

The results clearly show that the bleaching agents as contemplated herein with two chelating agents in preparation (A) exhibit a stronger lightening power than bleaching agents with one chelating agent in preparation (A).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth the the appended claims.

1. Agents for lightening keratinic fibres, comprising at least two separately packaged preparations (A) and (B) as well as an optional further preparation (C) which is packaged separately from preparations (A) and (B), wherein preparations (A), (B), and, when present preparation (C) are mixed immediately prior to use in order to form a mixture for application, wherein
i. preparation (A) comprises at least one persulphate and at least two chelating agents, and
ii. preparation (B) is free-flowing and comprises at least one oxidizing agent.

2. The agent according to claim 1, wherein that at least two chelating agents of preparation (A) are selected from the group of:
(a) nitrilotriacetic acid (NTA),
(b) diethylenetriamine pentaacetic acid (DTPA),
(c) ethylenediamine disuccinic acid (EDDS),
(d) ethylenediamine diglutaric acid (EDGA),
(e) 2-hydroxypropylenediamine disuccinic acid (HPDS),
(f) glycinamide-N,N′-disuccinic acid (GADS),
(g) ethylenediamine-N-N′-diglutaric acid (EDDG),
(h) 2-hydroxypropylenediamine-N-N′-disuccinic acid (HPDDS),
(i) ethylenediamine tetraacetic acid (EDTA),
(j) ethylenedicysteic acid (EDC),
(k) diaminoalkyl di(sulphosuccinic acid) (DDS),
(l) ethylenediamine-N-N′-bis(ortho-hydroxyphenyl acetic acid (EDDHA),
(m) N-2-hydroxyethyl-N,N-diacetic acid,
(n) glycerylimino diacetic acid,
(o) iminodiacetic acid-N-2-hydroxypropylsulphonic acid,
(p) aspartic acid-N-carboxymethyl-N-2,5-hydroxypropyl-3-sulphonic acid,
(q) α-alanine-N,N′-diacetic acid,
(r) aspartic acid-N,N′-diacetic acid,
(s) aspartic acid-N-monoacetic acid,
(t) dipicolinic acid,
(u) 1-hydroxyethane-(1,1-diphosphonic acid) (HEDP), or
(v) salts and derivatives thereof.

3. The agent according to claim 2, wherein as at least one of the chelating agents preparation (A) comprises a salt.

4. The agent according to claim 2, wherein as one of the chelating agents preparation (A) comprises 1-hydroxyethane-(1,1-diphosphonic acid) (HEDP).

5. The agent according to claim 2, wherein as at least two of the chelating agents preparation (A) comprises a salt of ethylene diamine tetraacetic acid (EDTA) and 1-hydroxyethane-(1,1-diphosphonic acid) (HEDP).

6. The agent according to claim 1, wherein preparation (A)—with respect to its weight—comprises the at least two chelating agents in a total quantity of from about 0.02% to about 12% by weight.

7. The agent according to claim 1, wherein preparation (A) comprises at least one persulphate selected from the group of ammonium peroxodisulphate potassium peroxodisulphate, and/or sodium peroxodisulphate.

8. The agent according to claim 1, wherein preparation (B), with respect to its weight, comprises as the at least one oxidizing agent hydrogen peroxide in quantities of from about 0.5% to about 30% by weight.

9. A method for lightening keratinic fibres, wherein at least two separately packaged preparations (A) and (B) are mixed together to form a mixture for application, the mixture is applied to the fibres, and after a treatment time the mixture rinsed out again, wherein preparation (A) comprises at least one persulphate and at least two chelating agents, and preparation (B) comprises at least one oxidizing agent fibres.

10. (canceled)

11. The agent according to claim 2, wherein as at least one of chelating agents preparation (A) comprises a salt of ethylene diamine tetraacetic acid (EDTA).

12. The agent according to claim 2, wherein as at least one of chelating agents preparation (A) comprises a sodium salt of ethylene diamine tetraacetic acid (EDTA).

13. The agent according to claim 1, wherein preparation (A)—with respect to its weight—comprises the at least two chelating agents in a total quantity of from about 0.2% to about 3% by weight.

14. The agent according to claim 1, wherein preparation (B), with respect to its weight, comprises as at least one oxidizing agent hydrogen peroxide in quantities of from about 6% to about 12% by weight.

15. The agent according to claim 1, wherein preparation (C) is present and wherein preparation (C) comprises at least one alkalizing agent.

16. The agent according to claim 15, wherein preparation (C) further comprises a silicon-containing compound.

17. The method according to claim 9, wherein a preparation (C) is further mixed together with preparations (A) and (B) to form the mixture, and wherein preparation (C) comprises at least one alkalizing agent.
